(19) United States
(12) Patent Application Publication
(10) Pub. No.: US 2009/0306198 A1
(43) Pub. Date: Dec. 10, 2009

(54) Anti-Oxidant Synergy Formulation Nanoemulsions to Treat Caner

(76) Inventors: Robert Nicolosi, Nashua, NH (US); Thomas Shea, Billerica, MA (US)

Correspondence Address: Carroll Peter G;Medlen & Carroll, 101 Howard Street Suite 350, San Francisco, CA, 94105, US

(21) Appl. No.: 11/991,991

(22) PCT Filed: Sep. 13, 2006
(86) PCT No.: PCT/US2006/035343

§ 371 Date: Jun. 15, 2009

Related U.S. Application Data
(60) Provisional application No. 60/717,702, filed on Sep. 16, 2005.

(51) Int. Cl. A61K 31/355; A61K 47/24; A61K 31/19; A61P 35/00; A61P 39/06

(52) U.S. Cl. 514/458; 514/785; 514/557

(57) ABSTRACT

A uniform microfluidized nanoemulsion is disclosed containing a synergistic combination of two antioxidants and a cell membrane stabilizer phospholipid (i.e., an anti-oxidant synergy formulation; ASF). The microfluidized nanoemulsion improves the combination's cell membrane permeability by at least four-fold over conventional nanoemulsion compositions, which significantly increases the intracellular concentration of typically cell-impermeant antioxidants (i.e., for example, tocopherol) and/or systemic bioavailability. As a nanoemulsion, synergistic combination has greater anticancer efficacy than the same combination applied as a free solution.

### FIELD OF INVENTION

The present invention relates to the field of cancer therapy. In one embodiment, the invention comprises a method to treat cancer using a uniform microfluidized nanoemulsion composition. In another embodiment, the composition comprises an anti-oxidant synergy formulation. In one embodiment, the composition comprises tocopherol. In one embodiment, the cancer comprises a solid tumor. In one embodiment, the cancer comprises a metastasized tumor mass.

### BACKGROUND OF THE INVENTION

Solid tumors arise in organs that contain stem cell populations. The tumors in these organs consist of heterogeneous populations of cancer cells that differ markedly in their ability to proliferate and form new tumors. In both breast cancers and central nervous system tumors, cancer cells differ in their ability to form tumors. While the majority of the cancer cells have a limited ability to divide, a population of cancer stem cells has an exclusive ability to extensively proliferate and form new tumors. Growing evidence suggests that pathways regulating a self-renewal of normal stem cells may be deregulated in cancer stem cells thereby resulting in a continuous expansion of self-renewing cancer cells and tumor formation. This suggests that agents that target the defective self-renewal pathways in cancer cells might lead to improved outcomes in the treatment of these diseases. Al-Hajj et al., “Self-renewal and solid tumor stem cells” Oncogene 23:7274-82 (2004). Currently, challenges regarding drug delivery to solid tumors are impeding progress in this field.

Drug delivery to solid tumors is one of the most challenging aspects in cancer therapy. Whereas agents seem promising during in vitro testing, clinical trials often fail due to unfavorable pharmacokinetics, poor delivery, low local concentrations, and limited accumulation in the target cell. One approach currently used in the art involves the treatment of solid tumors using tumor-associated vasculature targeting factors. These therapeutic regimens hope to reduce tumor progression by inhibiting tumor vascular development. tenHagen et al., “Solid tumor therapy: manipulation of the vasculature with TNF” Technol Cancer Res Treat 2:195-203 (2003). This approach fails, however, to directly provide a cytotoxic effect into the cancer cells themselves.

Neuroblastoma solid tumors, for example, are present in approximately 600 diagnosed cancer cases annually in the United States. Unfortunately, approximately 90% are children ≦5 years old. Screening programs of infants show that many cases escape detection because of spontaneous regression or maturation into benign lesions. Diagnosing a neuroblastoma usually requires CT (or HI), bone scan, metaiodobenzylguanidine (MIBG) scan, bone marrow tests, and urine catecholamine measurements. These procedures usually allow placement of a patient into a low-risk (90% survival) or high-risk (approximately 25%-30% survival) category. Patients, however, despite having a favorable clinical profile (e.g., localized tumor), are still likely to develop lethal metastatic disease. Kushner et al., “Neuroblastoma: a disease requiring a multitude of imaging studies” J Nucl Med. 45:1172-88 (2004).

Breast cancer also comprise solid tumors and is the most common female malignancy in most industrialized countries, as it is estimated to affect about 10% of the female population during their lifespan. Although its mortality has not increased along with its incidence, due to earlier diagnosis and improved treatment, it is still one of the predominant causes of death in middle-aged women. The primary treatment for breast cancer is surgery, either alone or combined with systemic adjuvant therapy (hormonal or cytotoxic) and/or post-operative irradiation. Approximately 25-30% of women with node-negative disease and at least 50-60% of women with positive nodes, who appear to be disease-free after loco-regional treatment, will relapse and need treatment for their metastatic disease. Thus, metastatic breast cancer is a significant and growing problem in oncology.

What is needed is a more effective cancer therapy method that: i) provides a composition having an improved cell membrane permeability, ii) provides an intracellular delivery of an anti-cancer agent; and iii) allows treatment of non-resectable and/or non-palpable tumors (i.e., for example, metastasized tumor cells).

### SUMMARY

The present invention relates to the field of cancer therapy. In one embodiment, the invention comprises a method to treat cancer using a uniform microfluidized nanoemulsion composition. In another embodiment, the composition comprises an anti-oxidant synergy formulation. In one embodiment, the composition comprises tocopherol.

In one embodiment, the cancer comprises a solid tumor. In one embodiment, the cancer comprises a metastasized tumor mass.

In one embodiment, the present invention contemplates a uniform nanoemulsion comprising a population of particles having maximum and minimum diameters, wherein the difference between said maximum and minimum diameters does not exceed 100 nm.

In one embodiment, the present invention contemplates a uniform nanoemulsion comprising an anti-oxidant formulation, wherein the formulation comprises a cell-impermeant anti-oxidant, a cell permeant anti-oxidant, and a phospholipid. In one embodiment, the nanoemulsion comprises a population of particles having diameters between approximately 10 and approximately 110 nanometers, wherein said nanoemulsion is not contaminated by particles having diameters larger than 110 nanometers. In one embodiment, the formulation comprises a pharmaceutical. In one embodiment, the formulation comprises a nutraceutical. In one embodiment, the cell-impermeant anti-oxidant comprises tocopherol. In one embodiment, the cell-permeant anti-oxidant comprises sodium pyruvate. In one embodiment, the phospholipid comprises phosphatidylcholine. In one embodiment, the formulation further comprises compound including, but not limited to, soybean oil, polysorbate 80, and HPLC grade water.

A nanoemulsion comprising tocopherol, wherein said nanoemulsion comprises a population of particles having diameters between approximately 10 and approximately 110 nanometers, wherein said nanoemulsion is not contaminated by particles having diameters larger than 110 nanometers.

In one embodiment, the present invention contemplates a method, comprising; a) providing; i) a subject, wherein said patient exhibits at least one cancer symptom; ii) a nanoemulsion comprising an anti-oxidant formulation, wherein said formulation comprises a cell-impermeant anti-oxidant, a cell permeant anti-oxidant, and a phospholipid; b) delivering said nanoemulsion to said patients under conditions such that said nanoemulsion penetrates a cell membrane and wherein said formulation is released intracellularly. In one embodiment, the nanoemulsion comprises a population of particles, wherein said particles having diameters between approximately 10 and approximately 110 nanometers, wherein said nanoemulsion is not contaminated by particles having diameters larger than 110 nanometers. In one embodiment, the cell membrane surrounds a normal cell. In one embodiment, the cell membrane surrounds a cancer cell. In one embodiment, the delivering comprises a method selected from the group consisting of oral, transdermal, intravenous, intraperitoneal, intramuscular, and subcutaneous. In one embodiment, the formulation comprises a pharmaceutical. In one embodiment, the formulation comprises a nutraceutical. In one embodiment, the cell-impermeant anti-oxidant comprises tocopherol. In one embodiment, the cell-permeant antioxidant comprises sodium pyruvate. In one embodiment, the phospholipid comprises phosphatidylcholine. In one embodiment, the formulation further comprises compound including, but not limited to, soybean oil, polysorbate 80, and HPLC grade water.

In one embodiment, the present invention contemplates a method, comprising; a) providing; i) a patient, wherein said patient exhibits at least one cancer symptom; ii) a nanoemulsion comprising an anti-oxidant formulation, wherein said formulation comprises a cell-impermeant anti-oxidant, a cell permeant anti-oxidant, and a phospholipid; b) delivering said nanoemulsion to said patients under conditions such that said at least one symptom is reduced. In one embodiment, the formulation comprises a population of particles encapsulating said compound, wherein said particles having diameters between approximately 10 and approximately 110 nanometers, wherein said nanoemulsion is not contaminated by particles having diameters larger than 110 nanometers. In one embodiment, the cancer symptom is caused by a neuroblastoma tumor. In one embodiment, cancer symptom is caused by a breast cancer tumor. In one embodiment, the delivering comprises intra-tumoral. In one embodiment, the delivering comprises a method selected from the group consisting of oral, transdermal, intravenous, intraperitoneal, intramuscular, and subcutaneous. In one embodiment, the cell-impermeant anti-oxidant comprises a tocopherol. In one embodiment, the cell permeant anti-oxidant comprises sodium pyruvate. In one embodiment, the phospholipid comprises phosphatidylcholine.

In one embodiment, the present invention contemplates a method, comprising; a) providing; i) a patient, wherein said patient exhibits at least one breast cancer symptom; ii) a nanoemulsion comprising an anti-oxidant formulation, wherein said formulation comprises a cell-impermeant anti-oxidant, a cell permeant anti-oxidant, and a phospholipid; b) delivering said nanoemulsion to said patients under conditions such that said at least one symptom is reduced. In one embodiment, the formulation comprises a population of particles encapsulating said compound, wherein said particles having diameters between approximately 10 and approximately 110 nanometers, wherein said nanoemulsion is not contaminated by particles having diameters larger than 110 nanometers. In one embodiment, the delivering comprises intra-tumoral. In one embodiment, the delivering comprises a method selected from the group consisting of oral, transdermal, intravenous, intraperitoneal, intramuscular, and subcutaneous. In one embodiment, the cell-impermeant anti-oxidant comprises a tocopherol. In one embodiment, the cell-permeant anti-oxidant comprises sodium pyruvate. In one embodiment, the phospholipid comprises phosphatidylcholine.

In one embodiment, the present invention contemplates a method, comprising; a) providing; i) a patient, wherein said patient exhibits at least one cancer symptom; ii) a uniform microfluidized nanoemulsion comprising tocopherol; b) delivering said nanoemulsion to said patients under conditions such that said at least one symptom is reduced. In one embodiment, the delivering comprises systemic. In one embodiment, the delivering comprises intra-tumoral. In one embodiment, the nanoemulsion further comprises a chemotherapeutic compound.

### DEFINITIONS

In general, the terms used herein are to be interpreted according to definitions generally accepted by those having ordinary skill in the art. Those listed below, however, are to be interpreted according to the following definitions.

The term “microfluidized”, “microfluidizing”, or “microfluidizer” as used herein refers to an instrument or a process that utilizes a continuous turbulent flow at high pressure including, but not limited to, a microfluidizer or other like device that may be useful in creating a uniform nanoemulsion. For example, microfluidizing may create a uniform nanoemulsion comprising a pharmaceutical, nutraceutical, or cosmeceutical from a premix within a thirty (30) second time frame (typically referred to a single pass exposure). Typically, a microfluidizer may be operated at a pressure of approximately 25,000 PSI to generate a uniform nanoemulsion.

The term “uniform nanoemulsion” as used herein, refers to any emulsion comprising any specified range of particle diameter sizes wherein the difference between the minimum diameter and maximum diameters do not exceed approximately 600 nm n, preferably approximately 300 nm, more preferably approximately 200=m, but most preferably approximately 100 nm (i.e., for example, microfluidization, as contemplated herein, produces a uniform nanoemulsion having a range of approximately 10-110 nm and is referred to herein as a uniform microfluidized nanoemulsion). Preferably, the total particle distribution (i.e., 100%) is encompassed within the specified range of particle diameter size. A particle diameter distribution where less than 3% is outside the specified range of particle diameter sizes is still contemplated herein as a uniform nanoemulsion.

The term “population” as used herein, refers to any mixture of nanoemulsion particles having a distribution in diameter size. For example, a population of nanoemulsion particles may range is particle diameter from between approximately 10-110 nm.

The term “nanoparticle” as used herein, refers to any particle having a diameter of less than 300 nanometers (nm), as defined by the National Science Foundation or preferably less than 100 nm, as defined by the National Institutes of Health. Most conventional techniques create nanoparticle compositions with an average particle diameter of approximately 300 nanometers (nm) or greater.

The term “compound” as used herein, refers to any pharmaceutical, nutraceutical, or cosmeceutical (i.e., for example, organic chemicals, lipids, proteins, oils, vitamins, crystals, minerals etc.) that are substantially soluble in a dispersion medium.

The term “chemotherapeutic compound” as used herein, refers to any pharmaceutical, nutraceutical, or cosmeceutical known to have either cytostatic or cytotoxic efficacy against cancerous cells.

The term “chemotherapeutic composition” as used herein, refers to any combination of chemotherapeutic compounds (i.e., for example, tamoxifen in combination with ASF). Other chemotherapeutic compounds include, but are not limited to, Alkeran, Cytoxan, Leukeran, Cis-platinum, BiCNU, Adriamycin, Doxorubicin, Cerubidine, Idamycin, Mithracin, Mutamycin, Fluorouracil, Methotrexate, Thioguanine, Toxotere, Etoposide, Vincristine, Irinotecan, Hycamptin, Matulane, Vumon, Hexylin, Hydroxyurea, Gemzar, Oncovin, and Etophophos.

The term “stable” as used herein, refers to any population of nanoemulsion particles whose diameters stay within the range of approximately 10-110 nm over a prolonged period of time (i.e., for example, one (1) day to twenty-four (24) months, preferably, two (2) weeks to twelve (12) months, but more preferably two (2) months to five (5) months). For example, if a population of nanoemulsion particles is subjected to prolonged storage, temperature changes, and/or pH changes whose diameters stay within a range of between approximately 10-110 nm, the nanoemulsion is stable.

The term “pharmaceutically acceptable” as used herein, refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salts” as used herein, refers to derivatives wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The term “therapeutically effective amount” as used herein, with respect to a drug dosage, shall mean that dosage that provides the specific pharmacological response for which the drug is administered or delivered to a significant number of subjects in need of such treatment. It is emphasized that ‘therapeutically effective amount,’ administered to a particular subject in a particular instance will not always be effective in treating the diseases described herein, even though such dosage is deemed a “therapeutically effective amount” by those skilled in the art. Specific subjects may, in fact, be “refractory” to a “therapeutically effective amount”. For example, a refractory subject may have a low bioavailability such that clinical efficacy is not obtainable. It is to be further understood that drug dosages are, in particular instances, measured as oral dosages, or with reference to drug levels as measured in blood.

The term “symptom is reduced” as used herein, refers to a qualitative or quantitative reduction in detectable symptoms, including, but not limited to, a detectable impact on the rate of recovery from disease (e.g. rate of tumor regression).

The term “refractory” as used herein, refers to any subject that does not respond with an expected clinical efficacy following the delivery of a compound as normally observed by practicing medical personnel.

The term “delivering” or “administering” as used herein, refers to any route for providing a pharmaceutical or a nutraceutical to a subject as accepted as standard by the medical community. For example, the present invention contemplates routes of delivering or administering that include, but are not limited to, oral, transdermal, intravenous, intraperitoneal, intramuscular, or subcutaneous.

The term “subject” or “patient” as used herein, refers to any animal to which an embodiment of the present invention may be delivered or administered. For example, a subject may be a human, dog, cat, cow, pig, horse, mouse, rat, gerbil, hamster etc.

The term “encapsulate”, “encapsulated”, or “encapsulating” refers to any compound that is completely surrounded by a protective material. For example, a compound may become encapsulated by a population of nanoemulsion particle formation during microfluidization.

The term “pharmaceutical” refers to any compound, natural or synthetic, used by those having skill in the medical arts to relieve at least one symptom of an abnormal medical condition (i.e., for example, injury or disease). For example, a patient having at least one cancer symptom may be delivered an anti-cancer pharmaceutical.

The term “nutraceutical” refers to any compound added to a dietary source (i.e., for example, a fortified food or a dietary supplement) that provides health or medical benefits in addition to its basic nutritional value.

The term “anti-oxidant formulation” refers to any mixture comprising a cell-impermeant anti-oxidant (i.e., for example, tocopherol), a phospholipid (i.e., for example, phosphatidylcholine), and a cell permeant anti-oxidant (i.e., for example, sodium pyruvate). Although it is not necessary to understand the mechanism of an invention, it is believed that when a nanoemulsion comprises the formulation the cell-impermeant and cell-permeant anti-oxidants are synergistic, thereby creating an “anti-oxidant synergy formulation”.

The term “cell-impermeant” as used herein, refers to any compound that is not cell membrane permeable to the extent that a therapeutically-effective amount of the compound is intracellularly delivered.

The term “cell-permeant” as used herein, refers to any compound that is cell membrane permeable to the exent that a therapeutically-effective amount of the compound is intracellularly delivered.

The term “phospholipid” as used herein, refers to any compound comprising a phosphoric ester of glycerol. Alternatively, other glycerol hydroxyl groups may be esterified to fatty acids. Phospholipids may include, but are not limited to, phosphatidylcholine, phosphatidylethanolamine, phosphatidylserine, phosphatidylinositol, phosphatidylglyerol, 3′-O-lyslyphosphatidylglycerol, or diphosphatidylglycerol (cardiolipin).

The term “anti-oxidant” as used herein, refers to a substance that, when present in a mixture or structure containing an oxidizable substrate biological molecule, inhibits oxidation or reactions promoted by oxygen and peroxides. Further, an antioxidant may quench singlet oxygen and free radical production. Antioxidants may include, but are not limited to, tocopherol and its derivatives, ascorbic acid, Vitamin C, Vitamin E, dietary antioxidants, phenylalanine, azide, p-phenylenediamine, n-propylgallate, diazabicyclo[2,2,2]octane, commercial reagents including, but not limited to, SlowFade and ProLong (Molecular Probes, Eugene Oreg.), sodium metabisulfite, gallic acid, alkyl gallates including, but not limited to, methyl gallate and ethyl gallate, butyl hydroxyanisole or nordihydroguararetic acid. The term “anti-oxidant” is not limited to compounds known to have strong reductive potentials (i.e., for example, Vitamin E) but also contemplates compounds that have relatively weaker reductive potentials (i.e., for example, Vitamin A, polyphenols, flavonoids, or beta-carotene and their derivatives).

The term “tocopherol” refers to any of several fat-soluble oily phenolic compounds with varying degrees of antioxidant vitamin E activity. In particular, a tocopherol may include, but is not limited to, α-tocopherol, β-tocopherol, γ-tocopherol or δ-tocopherol.

The term “membrane permeability” refers to the ability of any compound (i.e., hydrophilic or hydrophobic) to pass through a phospholipid bilayer cellular membrane. Such membrane structures are common in most biological tissues including, but not limited to, epithelial cells, breast cells, nerve cells, kidney cell, intestinal cells, etc.

The term “at risk for” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences.

The term “cell” as used herein, refers to any small usually microscopic mass of protoplasm bounded externally by a semipermeable membrane, usually including one or more nuclei and various nonliving products, capable alone or interacting with other cells of performing all the fundamental functions of life, and forming the smallest structural unit of living matter capable of functioning independently. For example, a cell as contemplated herein includes, but is not limited to, an epithelial cell, a breast cell, a nerve cell, a liver cell, a lung cell, a kidney cell etc. Further, cells as contemplated herein may include, but are not limited to, normal cells (i.e., non-cancerous cells) or transformed cells (i.e., cancerous cells).

### BRIEF DESCRIPTION OF THE FIGURES

FIG. 1 presents exemplary data showing the particle diameter distribution of a microfluidized plant sterol nanoemulsion population three (3) months after preparation.

FIG. 1A presents exemplary data showing the particle diameter distribution of a microfluidized plant sterol nanoemulsion three (3) months after preparation.

FIG. 2 presents exemplary data showing the particle diameter distribution of a microfluidized cod liver oil nanoemulsion population four (4) months after preparation.

FIG. 3 presents exemplary data showing the particle diameter distribution of a microfluidized tocopherol nanoemulsion population five (5) months after preparation.

FIG. 4 presents exemplary data showing the particle diameter distribution of a microfluidized ASF nanoemulsion population.

FIG. 5 presents exemplary data showing the effect of an intratumoral injection of a microfluidized ASF nanoemulsion on in vivo neuroblastoma in mice.

FIG. 6 presents exemplary data showing the effect of an intratumoral injection of a microfluidized ASF nanoemulsion on in vivo neuroblastoma in mice following pretreatment with a microfluidized nanoemulsion composition.

FIG. 7 presents exemplary data showing a comparison of apoptosis induction between microfluidized tamoxifen nanoemulsions and microfluidized ASF nanoemulsions.

FIG. 8 presents exemplary data showing a time course of 8-tocopherol plasma levels following improved membrane permeability by using a microfluidized nanoemulsion.

### DETAILED DESCRIPTION OF THE INVENTION

The present invention relates to the field of cancer therapy. In one embodiment, the invention comprises a method to treat cancer using a uniform microfluidized nanoemulsion composition. In another embodiment, the composition comprises an anti-oxidant synergy formulation. In one embodiment, the formulation comprises a tocopherol. In one embodiment, the cancer comprises a solid tumor. In one embodiment, the cancer comprises a metastasized tumor mass.

### I. Neuroblastoma

Neuroblastoma, the most common of all cancers found in children, may arise from a biochemical block of cellular differentiation and a resultant continuation of a proliferative state. Neuroblastoma often spontaneously reverts by undergoing partial differentiation and ultimate degeneration. In one embodiment, the present invention contemplates a useful therapeutic approach for clinical neuroblastoma comprising strategies to force neuroblastoma to differentiate. In one embodiment, the differentiation strategy comprises a reduction in intracellular reactive oxygen species.

Clinical and biologic features of this disease have been used to develop risk-based therapy approaches. Patients with low-risk disease can be treated with surgery alone. Patients with intermediate-risk features may survive after treatment with surgery and a relatively short course of standard dose chemotherapy. Unfortunately, most children with neuroblastoma present with advanced disease. More than 60% of patients with high-risk features will succumb to their disease despite intensive therapy including a myeloablative consolidation. Research efforts to understand the biologic basis of neuroblastoma and to identify new, more effective therapies are essential to improve the outcome for these children. Goldsby et al., “Neuroblastoma: evolving therapies for a disease with many faces” Paediatr Drugs. 6:107-22 (2004).

In one embodiment, the present invention contemplates a method to develop more effective neuroblastoma therapies. In one embodiment, the method comprises nude mice which are a recognized model for treatment of tumors. Although it is not necessary to understand the mechanism of an invention, it is believed that nude mice lack a fully-functional immune system and therefore do not mount a deleterious response against experimentally-induced tumors. Consequently, these mice may be a useful model system for analyses of the efficacy of anti-cancer treatments (i.e., for example, neuroblastoma solid tumors).

One known approach to treat neuroblastoma takes advantage of cell surface disialoganglioside over-expression. An immunoliposomal formulation covalently couples Fab′ fragments of the monoclonal antibody anti-GD(2) that is compatible with uptake systems in some neuroblastoma cell lines. When these immunoliposomes were loaded with either doxorubicin or the synthetic retinoid fenretinide some neuroblastoma cell proliferation inhibition was seen. Brignole et al., “Development of Fab′ fragments of anti-GD(2) immunoliposomes entrapping doxorubicin for experimental therapy of human neuroblastoma” Cancer Lett 197(1-2):199-204 (2003); and Raffaghello et al., “Immunoliposomal fenretinide: a novel antitumoral drug for human neuroblastoma” Cancer Lett 197(1-2):151-5 (2003).

Clinically, neuroblastoma usually presents as a malignant cancerous tumor in infants and children (1 out of 100,000, slightly more common in males) that develops from nerve tissue. The cause of neuroblastoma tumors is unknown. Neuroblastoma is most commonly diagnosed in children before age 5. The disorder occurs in approximately 1 out of 100,000 children and is slightly more common in boys. Neuroblastoma, however, can occur in many areas of the body and develops from the tissues that form the sympathetic nervous system (i.e., for example, exerting control over basic body functions, such as, but not limited to, heart rate, blood pressure, digestion, and levels of certain hormones). This tissue of origin for most neuroblastoma commonly begins in the abdomen from the tissues of the adrenal gland, but it may also occur in other areas. Metastasis may then involve the lymph nodes, liver, bones, and bone marrow.

Commonly seen symptoms with neuroblastoma patients, include, but are not limited to, pale skin, dark circles around the eyes, chronic fatigue (i.e., for example, excessive tiredness lasting for weeks to months), diarrhea, enlarged or swollen abdomen, abdominal mass, bone pain or tenderness, difficulty breathing, malaise (i.e., for example, general discomfort or uneasiness lasting for weeks or months), flushed or red skin, profuse sweating, tachycardia, uncontrollable eye movements, paralysis of the lower extremities, uncoordinated movement, irritability, or poor temper control.

Methods of neuroblastoma diagnosis may include, but are not limited to, computed tomography (CT) scans, magnetic resonance imaging (MRI) scans, chest X-rays, bone scans, bone marrow biopsy, hormone tests (i.e., for example, epinephrine), complete blood count (CBC), urine or blood catecholamine levels, or MBG scans.

Treatment common in the art varies depending on the location of the tumor, the extent of tumor spread and the age of the patient. In certain cases, surgery alone is enough, but often other therapies are needed. Anticancer medications (chemotherapy) may be recommended if the tumor is widespread. Radiation therapy may also be used.

The expected outcome varies. In very young children with neuroblastoma, the tumor may go away on its own, without any treatment, or the tissues of the tumor may mature and develop into a benign ganglioneuroma that can be surgically removed. In other cases, the tumor spreads rapidly. Response to treatment is variable. Treatment is often successful if the cancer has not spread, but if there has been spread to other areas, neuroblastoma is much harder to cure.

Complications may also occur during the course of neuroblastoma including, but not limited to, tumor metastasis, damage and loss of function of involved organ(s), kidney failure, liver failure, loss of blood cells produced by the bone marrow, decreased resistance to infection, or other organ system losses.

### III. Breast Cancer

Breast cancer is a malignant growth that begins in the tissues of the breast. Over the course of a lifetime, one in eight women will be diagnosed with one of several types of breast cancer. For example, ductal carcinoma begins in the cells lining the ducts that bring milk to the nipple and accounts for more than 75% of breast cancers. Another breast cancer type, lobular carcinoma, begins in the milk-secreting glands of the breast but is otherwise fairly similar in its behavior to ductal carcinoma. Alternatively, other varieties of breast cancer can arise from the skin, fat, connective tissues, and other cells present in the breast.

In one embodiment, the present invention contemplates treating patients at risk for breast cancer. Risk factors for breast cancer include, but are not limited to, age, gender, hormonal imbalance, family history, early menstruation, and late menopause, oral contraceptives (i.e., for example, birth control pills), hormone replacement therapy, obesity, alcohol consumption, exposure to pesticides and other industrial products, diethylstilbestrol (DES), radiation, previous cancer diagnosis or strong history of cancer in the family.

In one embodiment, the present invention contemplates treating patients exhibiting symptoms of breast cancer. Symptoms for breast cancer include, but are not limited to, a breast lump or mass usually painless, firm to hard and usually with irregular borders; lump or mass in the armpit; a change in the size or shape of the breast; abnormal nipple discharge (i.e., for example, bloody, clear-to-yellow, green fluid, or purulent); changes in the color or feel of the skin of the breast, nipple, or areola; change in appearance or sensation of the nipple; unilateral breast pain, enlargement, or discomfort; bone pain, weight loss, swelling of one arm, and skin ulceration

Upon breast cancer diagnosis, additional testing is usually performed, including chest X-ray and blood tests. Various initial treatments such as, but not limited to, surgery, radiation, chemotherapy, or a combination of these may then be recommended, not only for treatment, but also to help determine the stage of disease. Breast cancer development is measured by a staging process that is important to help guide future treatment and follow-up. Breast cancer stages are currently defined as:

STAGE 0. In situ disease in which the cancerous cells are in their original location within normal breast tissue. Known as, for example, DCIS (ductoral carcinoma in situ) or LCIS (lobular carcinoma in situ) this stage represents a pre-cancerous condition, and only a small percentage of DCIS tumors develop to become invasive cancers.

STAGE I. A tumor less than 2 cm in diameter without intrabreast metastasis.

STAGE IIA. A tumor 2 to 5 cm in size without intrabreast metastasis or a tumor less than 2 cm in size with intrabreast metastasis.

STAGE IIB. A tumor greater than 5 cm in size without intrabreast metastasis or a tumor 2 to 5 cm in size with intrabreast metastasis.

STAGE IIIA. A tumor smaller than 5 cm in size with intrabreast metastasis which are attached to each other or to other structures, or tumor larger than 5 cm in size with intrabreast metastasis.

STAGE IIIB. A tumor that has penetrated outside the breast to the skin of the breast or of the chest wall or has metastasized to lymph nodes inside the chest wall along the sternum.

STAGE IV. A tumor of any size with metastases beyond the region of the breast and chest wall, such as to liver, bone, or lungs (i.e., for example, systemic metastasis).

The choice of initial breast cancer treatment may be based on more than one factor. For stage I, II, or III cancers, the main considerations are to adequately treat the cancer and prevent a recurrence either at the place of the original tumor (local) or elsewhere in the body (metastatic). For stage 1V cancer, the goal is to improve symptoms and prolong survival. However, in most cases, stage 1V breast cancer cannot be cured.

Hormonal therapy with tamoxifen is used to block the effects of estrogen that may otherwise help breast cancer cells to survive and grow. Most women with breast cancer tumors producing estrogen or progesterone benefit from treatment with tamoxifen. A new class of medicines called aromatase inhibitors (i.e., for example, Aromasin®) have been shown to be as good or possibly even better than tamoxifen in women with stage 1V breast cancer.

Combination therapies are common treatments for many breast cancer patients. For stage 0 breast cancer, mastectomy or lumpectomy plus radiation is the standard treatment. However, there is some controversy on how best to treat DCIS. For stage I and II disease, lumpectomy (plus radiation) or mastectomy with at least “sentinel node” lymph node removal is standard treatment. Chemotherapy, hormone therapy, or both may be recommended following surgery. The presence of breast cancer in the axillary lymph nodes is very useful for staging and the appropriate follow-up treatment. Stage III patients are usually treated with surgery followed by chemotherapy with or without hormonal therapy. Radiation therapy may also be considered under special circumstances. Stage 1V breast cancer may be treated with surgery, radiation, chemotherapy, hormonal therapy, or a combination of these (depending on the situation). The clinical stage of breast cancer is the best indicator for prognosis (probable outcome), in addition to some other factors. Five-year survival rates for individuals with breast cancer who receive appropriate treatment are approximately, 95% for stage 0, 88% for stage I, 66% for stage II, 36% for stage III, and 7% for stage IV.

Even with aggressive and appropriate treatments, breast cancer often spreads (metastasizes) to other parts of the body such as, but not limited to, the lungs, liver and bones. The recurrence rate is about 5% after total mastectomy and removing armpit lymph nodes when the nodes are found not to have cancer. The recurrence rate is 25% in those with similar treatment when the nodes have cancer.

Despite improvements in breast cancer diagnosis (i.e., for example, early detection), about 1-5% of women with newly diagnosed breast cancer have a distant metastasis at the time of the diagnosis. In addition, approximately 50% of the patients primarily diagnosed with only a local disease eventually relapse with metastases. Eighty-five percent (85%) of these recurrences take place within the first five years after the primary manifestation of the disease. Breast cancer metastases may be found in nearly every organ of the body at autopsy. The most common sites of metastatic involvement observed are loco-regional recurrences in the skin and soft tissues of the chest wall, as well as in axilla, and supraclavicular area. The most common sites for distant metastasis include, but are not limited to, bone (30-40%), lung, and liver.

Metastatic breast cancer is generally considered to be an incurable disease. However, the currently available treatment options often prolong the disease-free state and overall survival rate, as well as increase the quality of the life. The median survival from the manifestation of distant metastases is about three years.

In some patients, advanced disease can be controlled with therapy for many years allowing good quality of life. This is particularly evident for those patients with hormone receptor positive disease and nonvisceral sites of metastases. It is contemplated that with better understanding of the molecular factors involved in the response to chemotherapy and increased efficiency of chemotherapy, regimens will substantially extend the survival for these patients, and in some patients, perhaps even extend survival to their otherwise natural life-span. However, despite these promises, the current reality is that treatment provides only temporary control of cancer growth for most patients with metastatic breast cancer. Consequently, in one embodiment, the present invention contemplates compositions and methods to deliver chemotherapeutic compounds to primary breast cancer tumors and metastases which provide more effective absorption of the chemotherapeutic compounds into a tumor cell. In order to provide the best options for treating and preventing metastases, in one embodiment, the present invention contemplates systemic administration of a uniform microfluidized nanoemulsion comprising a chemotherapeutic compound (i.e., for example, tamoxifen) or chemotherapeutic composition (i.e., for example, ASF).

Systemic drug therapy for advanced breast cancer is usually started with hormonal therapy due to its lower toxicity than the cytotoxic chemotherapies. The best candidates for hormonal therapy, based on their clinical features, are patients with a hormone receptor positive tumor (especially when both hormone receptors are positive), long term disease free survival, previous response to hormonal therapy, and non-visceral disease. Despite short second-line and even third-line responses to alternative hormonal therapies (e.g., second anti-estrogen or aromatase inhibitor) in advanced stage of breast cancer, nearly all patients finally become refractory to hormonal therapy and their disease progresses.

Due to its higher toxicity, cytotoxic chemotherapy is given to patients with disease refractory to hormonal therapy. In addition, it is frequently used as the first-line therapy for those with extensive visceral involvement of metastatic disease (e.g., lung or liver metastasis), with hormone receptor negative primary tumor, with extensive involvement of bone marrow, or with tumor that is so rapidly growing that the response to hormonal therapy can not be monitored. Combination chemotherapy for advanced breast cancer is generally considered more efficacious than single-agent therapy. In one embodiment, the present invention contemplates a uniform microfluidized nanoemulsion comprising a combination of a chemotherapeutic compound (i.e., for example, tamoxifen) and a chemotherapeutic composition (i.e., for example, ASF).

Advanced breast cancer is currently considered to be incurable and nearly all available chemotherapeutic drugs have been tested for use in its treatment. In embodiment, the present invention contemplates a uniform microfluidized nanoemulsion including, but not limited to, a chemotherapeutic compound or drug selected from the group comprising anthracyclines (which are topoII-inhibitors), doxorubicin, epirubicin, taxanes, paclitaxel, rapamycin, docetaxel, etoposide, amsacrine, and mitoxantrone.

Further, in some embodiment, the present invention contemplates similar chemotherapeutic compositions and methods using uniform microfluidized nanoemulsions for other cancerous diseases, including, but not limited to, lymphomas and leukemias.

In some embodiments, the present invention contemplates that chemotherapeutic compounds or compositions may be administered using nanoemulsions contemplated herein as adjuvant chemotherapy regimens (i.e., for example, administered in combination with either convention chemotherapy, radiotherapy or surgical intervention). For example whether given alone or combined with other cytotoxic drugs, the objective response rate to anthracyclines generally ranges from 40% to 80% in metastatic breast cancer. Although it is not necessary to understand the mechanism of an invention, it is believed that when anthracyclines are given using a uniform microfluidized nanoemulsion the metastatic breast cancer response rate would be significantly greater than 40-80%. It is further believed that, the rate of complete response would be greater than 5-15% and improving long term remission for longer than one to two years.

Currently, the proportion of patients who achieve complete, prolonged (i.e., several years) remissions is believed to be below 1%. More typically, these responses are partial (i.e., 50% reduction in tumor mass) and its duration ranges from 6 to 12 months. Thus, there is still a large number of patients who do not receive objective, clinical response to these cytotoxic drugs. In some embodiments, the present invention contemplates and methods of administering a uniform microfluidized nanoemulsions comprising chemotherapeutic compositions wherein prolonged remissions occur in 5-50% of patients, preferably 15-40% of patients, and more preferably in 20-30% of patients.

Therefore, there is a need to i) improve the systemic delivery of chemotherapeutic drugs or ii) administer chemotherapeutic compositions having an equal efficacy that are less toxic than those currently administered.

In some embodiments, the present invention contemplates methods of administering chemotherapeutic compounds effective against breast cancer encapsulated within a uniform microfluidized nanoemulsion. Nanoemulsions, as contemplated herein, have been demonstrated to have improved membrane permeability. See Example 8. These nanoemulsions may be given using any route of administration including, but not limited to, oral, transdermal, intravenous, intraperitoneal, intramuscular, intra-tumoral, or subcutaneous. It is specifically contemplated that a systemic administration of a uniform microfluidized nanoemulsion comprising a chemotherapeutic compound effective against breast cancer (i.e., for example, ASF, tamoxifen etc.) reduces the spread and growth of breast cancer metastases. Although it is not necessary to understand the mechanism of an invention, it is believed that systemically administered microfluidized nanoemulsions utilize their improved membrane permeability properties to intracellularly deliver chemotherapeutic compounds to the metastasized tumor cells. Alternatively, a local breast cancer solid tumor may received an intratumoral injection of a uniform microfluidized nanoemulsion comprising a chemotherapeutic compound.

### II. Nanoemulsion Production Techniques

Nanoemulsions have been generated by a variety of methods. In particular, these methods provide a wide variation in particle diameter and require organic solvents and or polymers. When these known nanoemulsions are considered for an oral drug or nutrient delivery system, issues of biocompatibility and physiological side effects become an important issue.

In one embodiment, the present invention contemplates a method of making a nanoemulsion comprising a continuous turbulent flow at high pressure. In one embodiment, the high pressure turbulent flow comprises microfluidization. In one embodiment, a uniform nanoemulsion is generated from a premix using a single pass exposure (i.e., for example, within a thirty (30) second time frame). In one embodiment, the uniform nanoemulsion comprises a population of particles whose difference between the minimum and maximum diameters does not exceed approximately 100 nm. In one embodiment, a uniform nanoemulsion is generated using a pressure of at least 25,000 PSI. In one embodiment, the present invention contemplates a method of making uniform microfluidized nanoemulsions without organic solvents or polymers. In one embodiment, the microfluidized nanoemulsion is made from a suspension. In another embodiment, the microfluidized nanoemulsion is made from a microemulsion.

In one embodiment, the present invention contemplates a uniform microfluidized nanoemulsion using compositions that are substantially soluble in a liquid dispersion medium. In one embodiment, the nanoemulsion encapsulates the compositions. In one embodiment, the compositions comprise a medical formulation. In another embodiment, the formulation is selected from the group comprising an anti-oxidant synergy formulation, a pharmaceutical formulation, or a nutraceutical formulation.

In one embodiment, the present invention contemplates a method of making a uniform microfluidized nanoemulsion comprising a population of particles whose diameter ranges from between 10-110 nm, without contamination of particle greater then 110 nm.

Microfluidization is a unique process that powers a single acting intensifier pump. The intensifier pump amplifies the hydraulic pressure to the selected level which, in turn, imparts that pressure to the product stream. As the pump travels through its pressure stroke, it drives the product at constant pressure through the interaction chamber. Within the interaction chamber are specially designed fixed-geometry microchannels through which the product stream will accelerate to high velocities, creating high shear and impact forces that generates a uniform nanoemulsion as the high velocity product stream impinges on itself and on wear-resistant surfaces.

As the intensifier pump completes its pressure stroke, it reverses direction and draws in a new volume of product. At the end of the intake stroke, it again reverses direction and drives the product at constant pressures, thereby repeating the process.

Upon exiting the interaction chamber, the product flows through an onboard heat exchanger which regulates the product to a desired temperature. At this point, the product may be recirculated through the system for further processing or directed externally to the next step in the process. Cook et al., “Apparatus For Forming Emulsions” U.S. Pat. No. 4,533,254 (1985); and Cook et al., “Method Of Forming A Microemulsion” U.S. Pat. No. 4,908,154 (1990)(both herein incorporated by reference).

Early attempts using microfluidizers to create nanoparticulate compositions required drug substances that were poorly soluble in a liquid dispersion medium. In one disclosed technology, “poorly soluble” was defined as less than 10 mg/ml. Bosch et al., “Process for preparing therapeutic compositions containing nanoparticles” U.S. Pat. No. 5,510,118 (1996)(herein incorporated by reference). While water-insolubility was preferably considered, oil-insoluble compounds were also subjected to a microfluidization process. Several others have implemented the basic '118 technology to encapsulate various insoluble compounds. In fact, these subsequent disclosures define a nanoparticle composition as “particles consisting of a poorly soluble therapeutic or diagnostic agent having adsorbed onto, or associated with, the surface thereof a non-crosslinked surface stabilizer”. Cooper et al., “Nanoparticulate Sterol Formulations And Novel Sterol Combinations” United States Patent Application Publication No. 2004/0033202 A1 (2004)(see pg 1 para 3)(herein incorporated by reference). Like the ' 118 patent, Cooper et al. discloses preparing nanoparticulate compositions using compounds that are poorly soluble in a liquid dispersion medium (i.e., water, oils, alcohols, glycols, etc.).

Two drugs that are insoluble in a selected liquid dispersion medium, meloxicam and topiramate, are suggested as potential candidates for improved clinical administration using the Cooper et al. nanoparticulate composition technology. Cooper et al., “Nanoparticulate meloxicam formulations” US Pat. Appln Publ. No. 2004/0229038 (2004); and Gustow et al., “Nanoparticulate topirarnate formulations” US Pat. Appl. Publ. No. 2004/0258758 (2004). Neither publication contains any exemplary data demonstrating the creation of a uniform microfluidized microemulsion having a particle diameter range of about 10-110 nm.

### III. Nanoemulsions As A Drug Delivery Platform

Encapsulation of therapeutic compounds for thermal, pH, or metabolic breakdown protection usually involves liposomes or other easily formed vesicles (i.e., a spontaneously forming oil-in-water emulsion). Nanoemulsions, in theory, may also provide protection for therapeutic compounds. In one embodiment, as detailed below, the present invention contemplates nanoemulsions that not only protect encapsulated compounds, but also improve intracellular drug delivery by promoting and facilitating drug transport through the plasma membrane.

Nanoemulsions have been considered as potential drug delivery platforms, in many different types of formulations and compositions. For example, a nanoemulsion formulation is described that requires a surfactant mixture component wherein the mixture has two or more surfactants (usually the first having a low hydrophilic-lipophilic balance and the second having a high hydrophilic-lipophilic balance). Roessler et al., “Nanoemulsion Formulations” United States Patent Application Publ No. 2002/0155084. Roessler et al. provides lengthy lists of potentially encapsulated compounds and nanoemulsion compositions. However, only compounds having specific skin permeation rates are discussed in any technical detail. Further, Roessler et al. teaches that the nanoemulsions created by the disclosed formulations form spontaneously and do not require high shear energies. Successful spontaneous formation of these nanoemulsions is dependent upon a complicated calculation involving surfactant densities and determination of the specific volume ratio's required. For example, the preferred nanoemulsion composition uses a 5:3 ratio of Span 80 to Tween 80 as the low and high hydrophilic-lipophilic balance surfactants, respectively.

Cosmetic formulations (i.e., those designed for topical application to the skin) are most effective when compounded as a cream, foam, or gel. These formulations are quite compatible with nanoemulsion technology. For example, dehydroepiandrosterone (DHEA) is known to be formulated as various emulsions containing various solubilizing and/or emulsifying agents. Besides the active compounds themselves, these compositions require a mixing of up to ten (10) specific ingredients that are responsible for the formation of the emulsion formulation during high pressure homogenization. Baldo et al., “Cosmetic Composition Containing A Steroid And A 2-Alkylalkanol Or An Ester Thereof” U.S. Pat. No. 6,486,147 (2002). Other simple emulsions are also described that may optionally, contain free-radical scavenger compounds in addition to the DHEA-derivatives. Dalko et al., “7-oxo-DHEA compounds for treating keratinous conditions/afflictions” U.S. Pat. No. 6,846,812 (2005).

Skin cancers have received some attention regarding using the above types of skin creams. Nanoemulsions containing 5-aminoevulinic acid are known that are intended for use in photodynamic therapy as well as in the photodiagnositic detection of proliferative cells. Schmid et al., “Nano-emulsion Of 5-Aminolevulinic Acid” U.S. Pat. No. 6,559,183 (2003). After homogenizing the various phases several times, the resulting particle size range was distributed between 200-10 nm. The basic nano emulsion carrier system used in Schmid et al. requires egg lecithin (i.e., 83% phosphatidylcholine), Polysorbatum 80, and Miglyol 812 (a triglyceride) and had been previously known. Weder et al., “Process for the production of a nanoemulsion of oil particles in an aqueous phase” U.S. Pat. No. 5,152,923 (1992). Weder et al. discloses high pressure homogenization of an aqueous lecithin/soybean oil premix, but only reports a particle size distribution of 100±30 nm (i.e., 70-130 mm).

Microemulsions and nanoemulsions have been briefly mentioned as possible carriers of specific diarylchroman derivatives for the treatment of various diseases. Included in the list of potential diseases are cancers such as, prostatic carcinoma, breast cancer, uterine cancer, cervical cancer, and colon cancer. Sangita et al., “(3R,4R)-Trans-3,4-diarylchroman derivatives and a method for the prevention and/or treatment of estrogen dependent diseases” United States Patent Application Publ No. 2005/0070597. Sangita et al. limit the technical details to liquid solutions and/or oral routes of administration and do not present any reasonable expectation of success for either making or using any micro or nanoemulsion formulations.

Other treatments for prostrate cancer are also known using nanoemulsion technology. Reduced cell proliferation and/or apoptosis is seen after intra-tumoral injection of mycobacterial DNA. Phillips et al., “Composition and method for inducing apoptosis in prostrate cancer cells” United States Patent No. 6,794,368 (2004). The preferred method of creating these emulsions uses sonication procedures to create average particle sizes of approximately 400 nm. Microfluidization techniques are only mentioned as possible (Model M-110Y, Microfluidics) and no attempts were apparently made to try this approach.

The use of nanoemulsions as a delivery system is generally directed to pharmaceutical formulations. Nanoemulsion nutraceutical formulation delivery, however, has received little attention. For example, one nanoemulsion system contains plant sterols. Bruce et al., “Method for producing dispersible sterol and stanol compounds” U.S. Pat. No. 6,387,411 (2002)(herein incorporated by reference). This technology, however, uses a grinding method to produce the nanoemulsions, and consequently, the particle diameter is at least six (6) times greater than contemplated herein. Although it is not necessary to understand the mechanism of an invention, it is believed that this diameter difference offers particular advantages in stability, efficacy, and penetrating cancer cell membranes (infra). Further, the '411 patent does not disclose the incorporation of absorbable micronutrients.

Exemplary nutraceuticals and dietary supplements are disclosed, for example, in Roberts et al., Nutriceuticals: The Complete Encyclopedia of Supplements, Herbs, Vitamins, and Healing Foods (American Nutriceutical Association, 2001), which is specifically incorporated by reference. Dietary supplements and nutraceuticals are also disclosed in Physicians' Desk Reference for Nutritional Supplements, 1st Ed. (2001) and The Physicians' Desk Reference for Herbal Medicines, 1st Ed. (2001), both of which are also incorporated by reference. A nutraceutical or dietary supplement, also known as a phytochemical or functional food, is generally any one of a class of dietary supplements, vitamins, minerals, herbs, or healing foods that have medical or biological effects on the body.

Exemplary nutraceuticals or dietary supplements include, but are not limited to, lutein, folic acid, fatty acids (e.g., DHA and ARA), fruit and vegetable extracts, vitamin and mineral supplements, phosphatidylserine, lipoic acid, melatonin, glucosamine/chondroitin, Aloe Vera, Guggul, amino acids (e.g., glutamine, arginine, iso-leucine, leucine, lysine, methionine, phenylalanine, threonine, tryptophan, and valine), green tea, lycopene, whole foods, food additives, herbs, phytonutrients, antioxidants, flavonoid constituents of fruits, evening primrose oil, flax seeds, fish and marine animal oils, and probiotics. Nutraceuticals and dietary supplements also include bio-engineered foods genetically engineered to have a desired property, also known as “pharmafoods.” In particular, these compounds include, but are not limited to, naturally occurring oils, fatty acids, and proteins. In one embodiment, a naturally occurring oil comprises fish oil (i.e., for example, cod liver oil). In one embodiment, a naturally occurring fatty acid comprises an omega-3 (i.e., for example, DHA). In one embodiment, the nanoemulsion comprises little or no fat. In one embodiment, a naturally occurring protein comprises soy or whey.

The present invention is directed to populations of nanoparticles or nanoemulsions comprising a delivery vehicle for all compounds whether absorbable nutrients including, but not limited to, fatty acids, carotenoids, tocopherois, tocotrienois, and coenzyme-Q; or non-absorbable, including, but not limited to, plant sterols, stanols, and triterpene alcohols (i.e., for example, oryzanol). Contemplated delivery methods, include but are not limited to oral, transdermal, intravenous, intraperitoneal, intramuscular, intra-tumoral, or subcutaneous. In another embodiment, the carotenoids include, but are not limited to, lutein and zeaxanthin. The present invention is also directed to populations of nanoparticles or nanoemulsions comprising an oral delivery vehicle for all non-absorbable (i.e., for example, fat soluble) plant sterol compounds including, but not limited to, phytosterols and phytostanols. In one embodiment, the compounds are mixed into a composition and encapsulated by nanoparticles or nanoemulsions. In one embodiment, common emulsifying agents are used to prepare a nanoemulsion. In one embodiment, the emulsifying agents include, but are not limited to, phospholipids, fatty acid monoglycerides, fatty acid diglycerides, or polysorbates.

In one embodiment, the present invention contemplates a composition comprising an anti-oxidant synergy formulation. In one embodiment, the anti-oxidant synergy formulation comprises tocopherol. In another embodiment, the anti-oxidant synergy formulation comprises sodium pyruvate. In another embodiment, the anti-oxidant synergy formulation comprises phosphatidylcholine.

The present invention also contemplates that certain nanoemulsion embodiments of the present invention comprise a surface-to-volume ratio that results in an improved membrane permeability over current methods and compositions known in the art.

### IV. Uniform Nanoemulsion Membrane Permeability

One of the prerequisites for the therapeutic action of a compound is its ability to penetrate lipid cell membranes. But in order to do this the drug must generally act through its undissociated, lipid soluble moieties. This chemistry, however, conflicts with the chemistry associated with drug dissolution and its ability to be administered orally or even parenterally. Some embodiments contemplated by the present invention avoid these conflicts by encapsulating anti-cancer formulations in such a manner that also facilitate their passage through a cell membrane (i.e., a tumor cell membrane or a non-tumor cell membrane).

Microemulsions have been reported as one possible carrier to address facilitated cell entry. These microemulsions are described as encapsulating hydrophobic drugs having a lipid core and stabilized by a monolayer of an amphipathic lipid (i.e., a phospholipid). Microemulsion stabilization is optimized by including a lipidized polymer that forms a matrix on the inner surface of the microparticles. Lu et al., “Artificial Lipoprotein Carrier System For Bioactive Materials” United States Patent Application Publ No. 2004/0234588. Lu et al. also describes nanoemulsions requiring a mixture of five (5) lipid components dissolved in chloroform. Following the evaporation of the organic solvent, the formulation was dissolved in a sodium chloride solution, sonicated and emulsified under pressure (70 psi, ten passes) to produce nanoparticles under 100 μm. One specific cholesterol-containing formulation utilized the cell membrane cholesterol uptake mechanism to facilitate intracellular entry of the formulation. Apparently, cholesterol transfer from the emulsion lipid core to a low-density lipoprotein (LDL) is required before this facilitated intracellular cholesterol entry occurs. Consequently, Lu et al. does not contemplate that the nanoparticles pass through a cell plasma membrane for intracellular delivery if an encapsulated drug.

The formation of a uniform mixture of predominantly small particles (i.e., for example, a population) may involve a physical process termed “emulsification”. An emulsion is traditionally defined in the art “as a system . . . consisting of a liquid dispersed with or without an emulsifier in an immiscible liquid usually in droplets of larger than colloidal size” Medline Plus Online Medical Dictionary, Merriam Webster (2005). Consequently, as the art developed emulsifiers capable of generating smaller and smaller diameter particles, the terms “microemulsion” and “nanoemulsion” became known. Conceptually, a microemulsion is one thousand-fold greater in diameter than a nanoemulsion. However, particle diameter distributions may vary widely in a non-controlled emulsification process creating considerable overlap between the nanoemulsion and microemulsion technologies.

In one embodiment, the present invention contemplates a premix comprising a compound substantially soluble (i.e., for example, greater than 30 mg/ml) in a liquid dispersion medium (i.e., for example, a heated liquid dispersion medium) and, optionally, common emulsifying agents including, but not limited to, phospholipids, fatty acid monoglycerides, fatty acid diglycerides, or polysorbates. In one embodiment, a nanoemulsion is created by exposing a premix to a continuous turbulent flow at a high pressure, wherein the pressure is at least 25,000 PSI. In one embodiment, the high pressure turbulent flow comprises microfluidization. In one embodiment, the nanoemulsion comprises particles encapsulating pharmaceutical and/or nutraceutical formulations. In one embodiment, the nanoemulsion comprises a uniform nanoemulsion having stable particles. In one embodiment, the microfluidization comprises a single pass exposure (i.e., for example, approximately thirty (30) seconds). In one embodiment, a uniform anti-oxidant synergy formulation microfluidized nanoemulsion has an improved anti-cancer efficacy. In other embodiments, the uniform microfluidized nanoemulsion further comprises a combination of an anti-oxidant synergy formulation and at least one conventional chemotherapeutic drug.

Although it is not necessary to understand the mechanism of an invention, it is believed that a much greater surface-to-volume ratio is reached in the uniform microfluidized nanoemulsion preparations made according to the present invention (i.e., for example, up to 6 fold) and results in greater stability. Consequently, it is further believed that, any incorporated pharmaceutical or nutraceutical has improved efficacy because of improved delivery (i.e., higher intracellular concentrations). It is further believed that nanoemulsions as contemplated by one embodiment of the present invention, when compared to known micron-sized micelles or microemulsions, have an improved delivery in to the intracellular space of a cell because of improved cell membrane permeability (i.e., for example, a tumor cell, or an epithelial cell). See Example 8. For example, it is known that pre-formed micron-size micelles containing plant stanols were up to three (3) times more efficacious in inhibiting cholesterol absorption than a suspension of crystalline stanol. Ostlund et al., “Sitostanol administered in lecithin micelles potently reduces cholesterol absorption in humans” Am J Clin Nutr 70:826-831 (1999).

An increased in vitro carotenoid bioavailability in cell cultures is observed when solubilizing the carotenoids in micelles. Xu et al., “Solubilization and stabilization of carotenoids using micelles: delivery of lycopene to cells in culture” Lipids 34:1031-1036 (1999). A disadvantage of using micelles, however, involves the use of chlorinated organic solvents, a practice that should be avoided in the processing of medical formulations. Another in vitro experiment demonstrates that a nanoemulsion preparation of lipophilic substances, such as fatty acids, vitamins, and beta-carotene can be delivered into cell culture medium (RPMI-1640) and incorporated by TK-6 cells. Zuelli et al., “Delivering lipophilic substances into cells using nanoemulsions” U.S. Pat. No. 6,558,941 (2003)(herein incorporated by reference).

Caroteinoids are known to have anti-cancer efficacy. The administration of liposomes containing caroteinoids was effective in a mouse model to prevent the metastasis of M5076 reticulosarcoma. Mehta et al., “Formulation And Use of Carotenoids In Treatment Of Cancer” U.S. Pat. No. 5,811,119 (1-998). The production of these liposomes required to use of organic solvents to dissolve the retinoid derivative in a phosphatidylcholine/soybean oil mixture followed by lyophilization and aqueous reconstitution.

In one embodiment, the present invention contemplates a nanoemulsion produced by a continuous turbulent flow at high pressure having improved cell membrane permeability properties when compared to conventional nanoparticulate compositions and/or nanoemulsions currently known in the art. It is known that nanoparticles deliver and/or release drugs (i.e., for example, norflaxin) and/or proteins (i.e., for example, serum albumin) more effectively than ricroparticles. Jeon et al., “Effect of solvent on the preparation of surfactant-free poly(DL-lactide-co-glycolide) nanoparticles and norfioxacin release characteristics” Int J Pharm 207; 99-108 (2000); and Panyam et al., “Polymer degradation and in vitro release of a model protein from poly(D,L-iactide-co-glycolide) nano- and microparticles” J Control Release 92:173-187 (2003).

One embodiment of the present invention contemplates a uniform microfluidized nanoemulsion having improved membrane permeability when compared to conventional nanoparticulate compositions and/or nanoemulsions currently known in the art. One advantage of uniform microfluidized nanoemulsions comprises a specific (i.e., for example, narrow) particle diameter range (i.e., for example, 10-110 nm). Most conventional nanoparticle compositions and/or nanoemulsions currently known have a wide distribution of particle diameters that interfere with the improved efficacies and membrane permeability of the smaller sized particles.

In one embodiment, the present invention contemplates a microfluidized nanoemulsion (i.e., for example, 40-60 nm sized particles) having an improved delivery of an antioxidant (i.e., for example, tocopherol) to cells (i.e., for example, epithelial cells, breast cancer cells, or neuroblastoma cells) over a traditional ricroemulsion.

The present invention has solved the problem of generating nanoemulsions with highly variable particle diameters and provides a more uniformly small-sized nanoemulsions (i.e., for example, a uniform nanoemulsion comprising stable particles). Consequently, these uniform nanoemulsions provide improved membrane permeability when compared to conventional nanoparticle compositions and/or nanoemulsions currently known in the art independent of the mode of delivery which includes, but is not limited to, oral, transdermal, intravenous, intraperitoneal, intramuscular, intra-tumoral, subcutaneous, etc.

### V. Anti-Oxidant Synergy Formulations (ASF)

A synergistic effect of a combination of tocopherol, phosphatidylcholine, and sodium pyruvate (i.e., ASF) was initially observed to promote wound healing. Bauer et al., “Reversal of doxorubicin-impaired wound healing using triad compound” Am Surg 60:455-459 (1994); Martin A., “The use of antioxidants in healing” Dermatol Surg 22:156-160 (1996); and Martin et al., “Evaluation of CRT healing components in two different topically-treated cold sore animal models” Antiviral Res 26:206 (1995). Further studies demonstrated the protective effects of ASF in neuronal cell culture and in situ central nervous system tissue when exposed to altered states of oxidation. Shea et al., “Efficacy of vitamin E, phosphatidyl choline, and pyruvate of buffering neuronal degeneration and oxidative stress in cultured cortical neurons and in central nervous tissue of apolipoprotein E-deficient mice” Free Rad Biol Med 33:276-282 (2002). Generally, ASF resulted in overall improved neuronal health represented by improved sprouting beyond that observed in the presence of serum alone (i.e., increased proliferative capacities).

It has been suggested that the glutathione redox system and vitamin E may be interdependent as protective agents during oxidative stress. For example, following chemical oxidant-induced depletion of intracellular glutathione, cell morphology and viability are maintained by the continuous presence of cellular alpha-tocopherol above a threshold level of 0.6-1.0 nmol/10⁶ cells. α-Tocopherol threshold-dependent cell viability may be directly correlated with the prevention of the loss of cellular protein thiols in the absence of intracellular glutathione. Although it is not necessary to understand the mechanism of an invention, it is believed that one potential mechanism for this phenomenon may include a direct reductive action of α-tocopherol on protein thiyl radicals, and the prevention of oxidation of protein thiols by scavenging of lipid peroxyl radicals by α-tocopherol. Pascoe et al., “Cell calcium, vitamin E, and the thiol redox system in cytotoxicity” Free Radic Biol Med. 6(2):209-24 (1989).

Although it is not necessary to understand the mechanism of an invention, it is believed that while in free solution tocopherol can prevent oxidative damage to a plasma membrane. The lipophilic nature of tocopherol, however, restricts its ability to quench cytosolic oxidizing compounds (i.e., has cell impermeant qualities). It is further believed that while in free solution pyruvate, which is cell permeant, quenches intracellular oxidative species that may be inaccessible to tocopherol. In one embodiment, the present invention contemplates a composition comprising an ASF-encapsulated nanoemulsion that solves this problem of a cell impermeant anti-oxidant, such as tocopherol, from being intracellularly inaccessible. In one embodiment, a nanoemulsion intracellularly delivers both a cell impermeant (i.e., for example, tocopherol) and a cell permeant (i.e., for example, pyruvate or N-acetyl cysteine), thereby providing a synergistic anti-oxidant effect. It is further believed that phosphatidylcholine may provide stability to the plasma membrane, thereby providing cell survival following oxidative membrane damage. For example, phosphatidylcholine provides a source of fatty acids for membrane stabilization and repair, and, in doing so, obviates necessary phosphatidylcholine synthesis steps that themselves generate reactive oxygen species. Alternatively, an ASP phospholipid may be substituted with an emulsifier including, but not limited to, ethoxylated monoglycerides, polysorbates (i.e., for example, 60, 65, or 80), sorbitan monostearate, sodium steroyl lactalate (i.e., Emplex®), calcium steroyl lactylate, diacetyl tartaric acid ester of monoglycerides (DATEM), or hard fat and soft fat derived distilled monoglycerides.

Reactive oxygen species (ROS) may result from cell metabolism as well as from extracellular processes. ROS may also exert some functions necessary for cell homeostasis maintenance. When produced in excess, however, they can play a role in the causation of cancer. ROS mediated lipid peroxides are known to participate in chain reactions that amplify damage to biomolecules including DNA. This DNA attack gives rise to mutations that may involve tumor suppressor genes or oncogenes, and this is an oncogenic mechanism.

On the other hand, ROS production may also be used as a cancer therapy. For example, ROS production is one mechanism shared by many chemotherapeutic drugs known to induce cellular apoptosis. Although it is not necessary to understand the mechanism of an invention, it is believed that the particular cancer-related ROS mediated cell response depends upon the duration and intensity of cellular exposure to an ROS-enriched environment. Thus, the intracellular redox status may control oncogenesis and also may dictate tumor susceptibility to specific chemotherapeutic drugs. Cejas et al., “Implications of oxidative stress and cell membrane lipid peroxidation in human cancer” Cancer Causes Control. 15:707-19 (2004).

It is known that transformed cell-derived ROS may exhibit directed and specific signaling functions, some of which are beneficial and some of which can become detrimental to transformed cells. Bauer G., “Signaling and proapoptotic functions of transformed cell-derived reactive oxygen species” Prostaglandins Leukot Essent Fatty Acids 66:41-56 (2002). On one hand, it has been suggested that a membrane associated NADPH oxidase produces extracellular superoxide anions that exhibit transmembrane signaling functions to regulate proliferation and maintain the transformed state. On the other hand, when superoxide anions are intracellularly dismutated into hydrogen peroxide predisposing the tumor cells to apoptotic mechanisms or interact with natural antitumor system cells (i.e., fibroblasts, granulocytes, and macrophages).

However, despite a commonly art-recognized convention that ROS is harmful to cells, new data and theories lend credence to the hypothesis that ROS may also play a role as a signaling molecule. For example, a low level of intracellular ROS is believed linked to proliferation and cell cycle progression. This idea provides an explanation for observations that a pro-oxidant state (i.e., for example, elevated intracellular ROS) may be associated with the transformed cells (i.e., cancer cells). Perivaiz et al., “Tumor intracellular redox status and drug resistance—serendipity or a casual relationship? Curr Pharm Des 10:1969-1977 (2004). These elevated intracellular ROS levels in tumor cells are also thought to cause resistance to: i) the activation of some apoptotic cell death receptor complexes (i.e., for example, Fas) or, ii) chemotherapeutic pharmacological activity.

In one embodiment, an anti-oxidant synergy formulation comprises tocopherol. In one embodiment, the formulation further comprises phosphatidylcholine. In one embodiment, the formulation further comprises sodium pyruvate. In one embodiment, the formulation further comprises compound including, but not limited to, soybean oil, polysorbate 80, and HPLC grade water. In one embodiment, the formulation further comprises a chemotherapeutic drug (i.e., for example, tamoxifen).

Although it is not necessary to understand the mechanism of an invention, it is believed that ASF reduces the generation of reactive oxygen species (ROS), lessens cellular toxicity. It is further believed that ASP promotes axonal elaboration in neurons thereby promoting neuronal health by: i) preventing proliferation; and ii) promoting differentiation. This hypothesis suggests the possibility that ASF, with proper administration, may foster differentiation (and therefore ultimate degeneration) of most any solid tumor, and may therefore represent a novel treatment approach towards many incurable cancer diseases.

It should be noted that ASF, when administered as a free solution, was unable to prevent continued increase in size of tumors generated following injection of neuroblastoma into nude mice, despite injection directly into the tumor. See Example V. Since ASF is effective on these cells in culture in the presence of serum, one likely interpretation is that in vivo an insufficient concentration of ASF was maintained at the tumor site such that membrane absorption occurred or that the ASP is unable to efficiently able to pass the membrane barrier. In one embodiment, the present invention contemplates a microfluidized nanoemulsion comprising ASF that arrests tumor growth. In one embodiment, tumor growth is arrested within twenty-four hours of delivery. In another embodiment, tumor growth is reversed, wherein tumor shrinkage is observed.

### EXPERIMENTAL

The following examples are specific embodiments as contemplated by the present invention and are not intended to be limiting.

### Example 1

### Stable Formulation of Plant Sterol Microfluidized Nanoemulsions

This example presents one plant sterol embodiment of a microfluidized nanoemulsion. The step-wise procedure is as follows:

1. Heat 4 g of soybean oil

2. Add 5 g soy lecithin, stir and heat to 90° C.

3. Add 1 g plant sterol, stir and heat 10 mins

4. Add 250 mg polysorbate 80.

5. Heat 240 mL de-ionized water to 70° C.

6. Add step 4 mixture to step 5 mixture, keep stir bar and heat on for 30 mins

7. Homogenize step 6 mixture for 24 mins

8. Stir formulation for 10 mins on hot plate

9. Microfluidize using a M-110EH unit once at 25,000 PSI

10. Do particle diameter analysis using a Malvern Nano S instrument

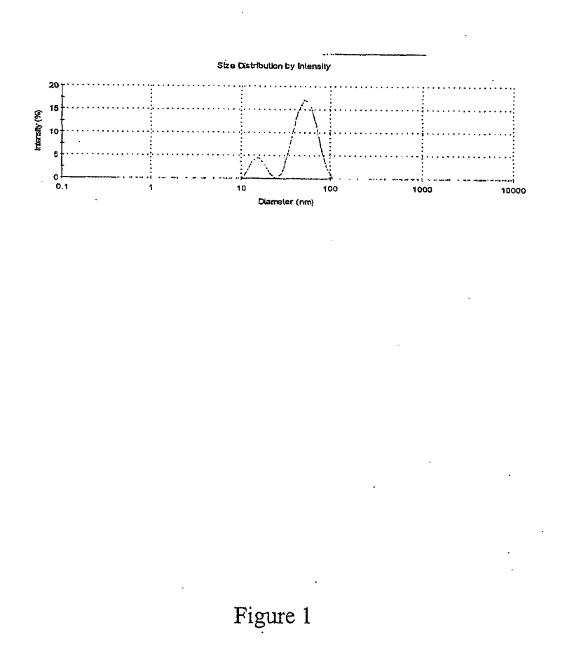

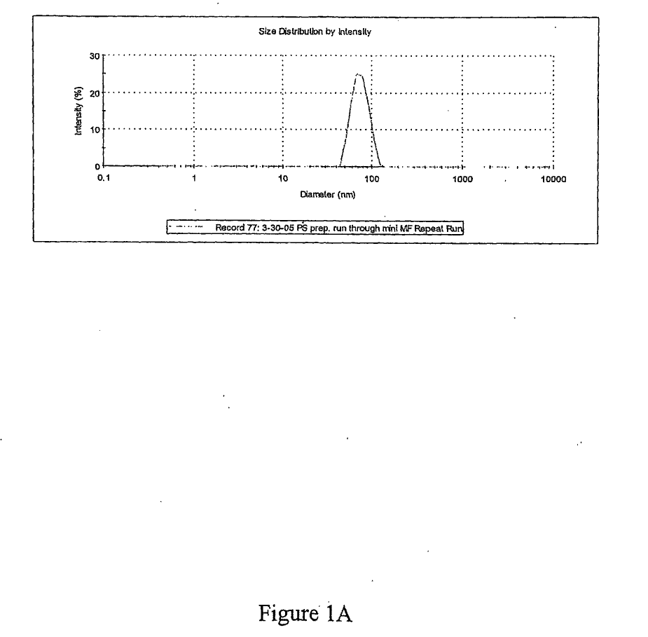

The mean particle diameter (i.e., Peak 1/Peak 2) for these microfluidized plant sterol nanoemulsions was 39 nm. See FIG. 1. The average particle diameter data for the plant sterol microfluidized nanoemulsion is shown in Table 2 below.

TABLE 2
Microfluidized Plant Sterol Nanoemulsion

| | Diam. (nm) | % Intensity | Width (nm) |
|---|---|---|---|
| Peak 1: | 54.16 | 85.86 | 14.36 |
| Peak 2: | 15.55 | 14.14 | 2.521 |
| Peak 3: | 0 | 0 | 0 |

Z-Average: 38.91; PDI: 0.228; Intercept: 0.9764.

After three months the particle diameter was again determined. The mean particle diameter (i.e., Peak 1) for this microfluidized plant sterol nanoemulsion was 64.4 nm. See FIG. 1A. The average particle diameter data for the three month plant sterol nanoemulsion is shown in Table 3 below.

TABLE 3
Three Month Storage: Microfluidized Plant Sterol Nanoemulsion

| | Diam. (nm) | % Intensity | Width (nm) |
|---|---|---|---|
| Peak 1: | 74.8 | 100 | 120.8 |
| Peak 2: | 0 | 0 | 0 |
| Peak 3: | 0 | 0 | 0 |

Z-Average: 64.4; PDI: 0.196; Intercept: 0.969.

### Example 2

### Stable Formulation of Cod Liver Oil Microfluidized Nanoemulsions

This example presents one cod liver oil embodiment of a microfluidized nanoemulsion that has a stable particle diameter for at least four months. The step-wise procedure is as follows:

1. Heat 5 g of soybean oil (65° C.)

2. Add 5 g cod liver oil, stir and heat to 80° C.

3. Add 6 g polysorbate 80, stir and heat 20 mins

4. Add 200 mL de-ionized water, stir and heat 30 mins

5. Microfluidize using a M-110EH unit once at 25,000 PSI

6. Do particle diameter analysis using a Malvern Nano S instrument

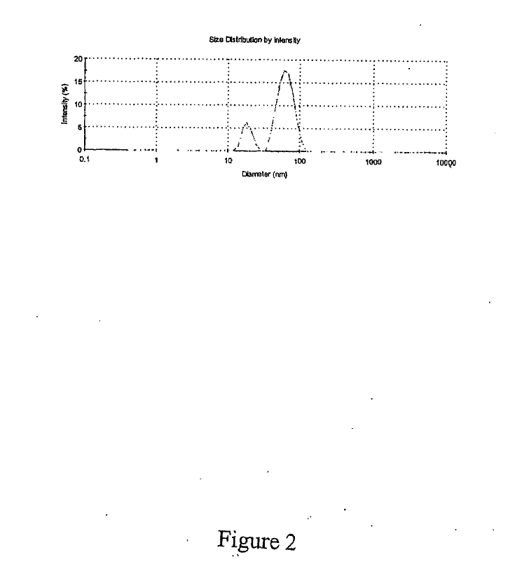

The mean particle diameter (i.e., Peak 1/Peak 2) for this cod liver oil microfluidized nanoemulsion was 58 nm. Before microfluidization, the mean particle diameter of the cod liver oil suspension was 2,842 nm. This represents a 50-fold reduction with a single pass through the microfluidizer. Four months after the microfluidization process, the particle diameter was again determined and found not to have changed. See FIG. 2. The average particle diameter data from the four-month microfluidized sample is presented in Table 4.

TABLE 4
Microfluidized Cod Liver Oil Nanoemulsion
Four Months After Preparation

| | Diam. (nm) | % Intensity | Width (nm) |
|---|---|---|---|
| Peak 1: | 63.92 | 82.22 | 15.62 |
| Peak 2: | 18.51 | 17.78 | 2.771 |
| Peak 3: | 0 | 0 | 0 |

Z-Average: 45.15; PDI: 0.247; Intercept: 0.9707.

### Example 3

### Stable Formulation of Tocopherol Microfluidized Nanoemulsions

This example presents one tocopherol embodiment of a microfluidized nanoemulsion that maintains particle diameter for at least five months. The step-wise procedure is as follows:

1. Heat 13.5 g of soybean oil

2. Add 2 g tocopherol, stir and heat to 90° C.

3. Heat 2 g polysorbate 80 in 100 mL de-ionized water, heat to 75° C.

4. Add step 3 mixture to step 2 mixture

5. Heat 300 mL di-ionized water and 6 g polysorbate 80, heat till 70° C.

6. Add step 4 mixture to step 5 mixture, keep stir bar and heat on

7. Homogenize step 6 mixture for 2-4 mins

8. Stir formulation for 3-5 ml's on hot plate

9. Microfluidize using a M-110EH unit once at 25,000 PSI

10. Do particle diameter analysis using a Malvern Nano S instrument

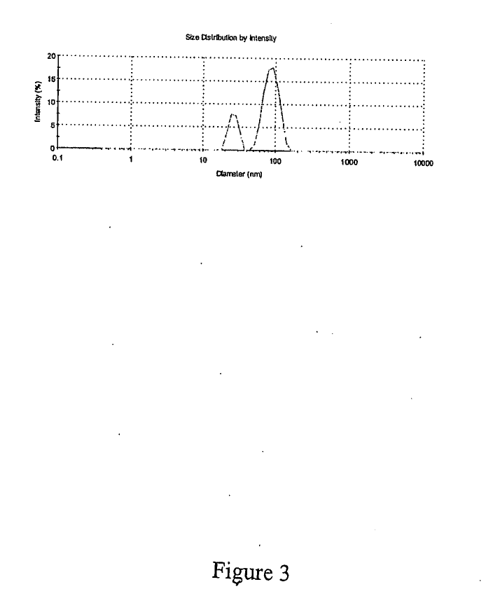

The mean particle diameter for the tocopherol microfluidized nanoemulsion was 64 nm. Before microfluidization, the mean particle diameter for the tocopherol suspension was 1,362 nm. This represents a 21-fold reduction a single pass through the microfluidizer. Five months after the microfluidization process, the particle diameter was again determined and found not to have changed. See FIG. 3. The average particle diameter data from the five-month microfluidized sample is presented in Table 5.

TABLE 5
Microfluidized Tocopherol Nanoemulsion
Five Months After Preparation

| | Diam. (nm) | % Intensity | Width (nm) |
|---|---|---|---|
| Peak 1 | 88.06 | 77.84 | 19.99 |
| Peak 2 | 26.46 | 22.16 | 3.651 |
| Peak 3 | 0 | 0 | 0 |

Z-Average: 58.07; PDI: 0.234; Intercept: 0.9697

### Example IV

### Preparation of an ASF Nanoemulsion

This example presents one embodiment of an ASF nanoemulsion proven effective against cancer cells. The step-wise procedure is as follows:

1. Heat 10 g of soybean oil while stirring.

2. Add phosphatidylcholine to a final concentration of 0.44 mg/ml.

3. Add sodium pyruvate to a final concentration of 2.14 mg/ml.

3. Add tocopherol to a final concentration of 2.06 mg/ml.

4. Add 10 g polysorbate 80.

5. Add HPLC-grade water (final volume=121.3 ml)

6. Maintain stirring for 15-20 minutes.

7. Homogenize step 6 mixture for 30 seconds

8. Microfluidize using a M-110EH unit once at 25,000 PSI

9. Do particle diameter analysis using a Malvern Nano S instrument

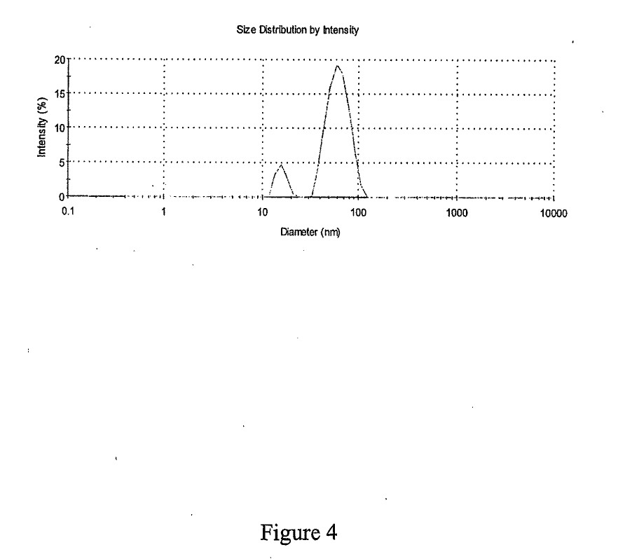

The mean particle diameter for the ASF microfluidized nanoemulsion was 47 nm (Peak I/Peak II). See FIG. 4. The average particle diameter data from the five-month microfluidized sample is presented in Table 6.

TABLE 5
Microfluidized ASF Nanoemulsion

| | Diam. (nm) | % Intensity | Width (nm) |
|---|---|---|---|
| Peak 1 | — | 88.71 | — |
| Peak 2 | — | 11.29 | — |

Z-Average: 46.97

### Example V

### ASF In Vivo Neuroblastoma Tumor Treatment

This example presents data regarding one embodiment of ASF anti-cancer efficacy using an in vivo neuroblastoma tumor mouse model.

In vivo tumors were generated by injecting tumor cells into thirty (15) nude mice (formerly C57BL/6J-Hfh11nu, now referred to as B6.AKR/J-Foxnlnu; Jackson Laboratories) by subcutaneous injection of mouse NB2a/d1 neuroblastoma cells (300,00 cells/mL). The generated tumors were observed to grow dramatically for 9-10 days.

ASF was prepared in accordance with Example IV. The injected mice were divided into three groups comprising approximately 4-5 mice per group.

During the first five (5) days of tumor growth, microfluidized ASF (Group 1) was injected directly into the tumors immediately after initial detection. Controls included injection of an ASF premix group (i.e., without microfluidization; Group II)) and an untreated group (Group III). Groups I and II were injected with a 20 μl volume into an approximately sized 0.2 mm diameter tumor.

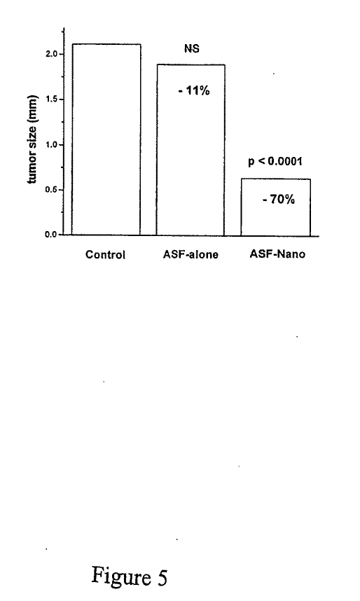

Two independent experiments were performed with similar results. The results for the pooled data (i.e., 8-10 mice per group) are shown in FIG. 5. While ASF premix injection did little to reduce tumor size, the microfluidized ASF nanoemulsion reduced tumor size by approximately 70%. This represents an approximate 6-fold improved efficacy.

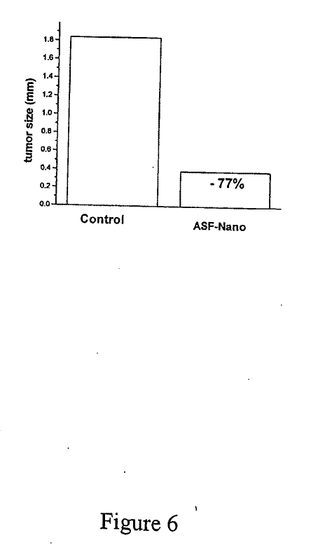

In a separate experiment, the mice were injected intraperitoneally with the microfluidized ASF nanoemulsion on the day before tumor cell injection. When a tumor was detected (i.e., approximately 0.2 mm diameter), an intra-tumoral injection of microfluidized ASF nanoemulsion was given (approximately 20 μl). The data show that this prophylactic treatment paradigm reduced tumor diameter sizes by approximately 77%. See FIG. 6.

This data indicates that tumor growth maybe arrested when given an intra-tumoral injection of microfluidized ASF nanoemulsion corresponding to approximately 2 mm² of surface area. In one embodiment, the present invention contemplates larger tumors may respond in a similar manner using injections at multiple sites (i.e., those corresponding to 2 mm² of tumor surface area).

### Example VI

### In Vitro Breast Cancer Comparative ASF Therapy

This example presents exemplary data shows the equivalent effectiveness of microfluidized nanoemulsion ASF preparations and microfluidized nanoemulsion tamoxifen preparations prepared in accordance with Example 4.

Human breast cancer cell lines MCF-7 and HTB-20 were subcultured in flasks accompanied by daily changes of medium comprising RPMI 1640 supplemented with fetal bovine serum (10%, v/v). The cultures were maintained in a humidified chamber at 37° C. with 5% CO₂ and 95% air. The cells were harvested at confluency by trypsinzation and suspended at a cell density of 200,000 to 300,000 cells/ml in a small volume of the growth medium to be used.

In a typical experiment in which the effects of the anti-cancer nanoemulsions on cell growth were to be examined, aliquots of the cell suspension containing about 20,000 to 30,000 cells were replicately plated in dishes containing 3 ml of the growth medium consisting of RPMI 1640 supplemented with charcoal-stripped fetal bovine serum (5%, v/v) and of penicillin-streptomycin (10 units and 10 μg, respectively) and mycostatin (2.5 units/ml) and incubated at 37° C. in the humidified chamber. On day 1 when the cell adhered firmly to the bottom surface of the dishes, the medium was aspirated off and the cells then challenged by the anti-cancer nanoemulsions; groups of replicate dishes received 3 ml of fresh growth medium containing either ASF or tamoxifen at the indicated concentrations. The control groups received only the microfluidized nanoemulsion, and ASF or tamoxifen in free solution.

At various time intervals (i.e., Days 1-17), triplicate dishes were withdrawn for cell counting; after removal of the medium by aspiration, the adhered cells were washed twice with 1 ml of Eagles balanced salt solution without Ca²⁺ and Mg²⁺ and dislodged from the dish by trypsinization involving the incubation of 0.5 ml of trypsin-EDTA solution (1×) for 5 minutes at 37° C., after which the protease reaction was stopped by adding 2.5 ml of a “stop” medium consisting of RPMI 1640 and 5% fetal calf serum. The cells were then collected by centrifugation with subsequent suspension in the medium. An aliquot of the suspension was subjected to cell counting using a Coulter Counter (data expressed as 10⁶ cells per dish).

The data show that the time course of tamoxifen-induced apoptosis of both MCF-7 and HTB-20 cells was improved from 2-10 fold when applied as a microfluidized nanoemulsion. See Tables 6 & 7.

TABLE 6
Effect Of Microfluidized Tamoxifen On HTB-20 Cell Culture Growth

| | Microfluidized | | Microfluidized |
|---|---|---|---|
| Days Of | Nanoemulsion | Tamoxifen In Free | Tamoxifen |
| Incubation | Control | Solution | Nanoemulsion |
| 1 | 5 | 4 | 2 |
| 3 | 26.5 | 12 | 4.5 |
| 5 | 37 | 24 | 5 |
| 7 | 46 | 28 | 5 |
| 10 | 90.5 | 41 | 6 |
| 12 | 149.5 | 51 | 4 |

TABLE 7
Effect Of Microfluidized Tamoxifen On MCF-7 Cell Culture Growth

| | Microfluidized | | Microfluidized |
|---|---|---|---|
| Days Of | Control | Tamoxifen In Free | Tamoxifen |
| Incubation | Nanoemulsion | Solution | Nanoemulsion |
| 5 | 7 | 5 | 5 |
| 8 | 47 | 19 | 8 |
| 10 | 59 | 25 | 10 |
| 12 | 74 | 33 | 10 |
| 15 | 86.5 | 50 | 13 |
| 17 | 98 | 67.5 | 14 |

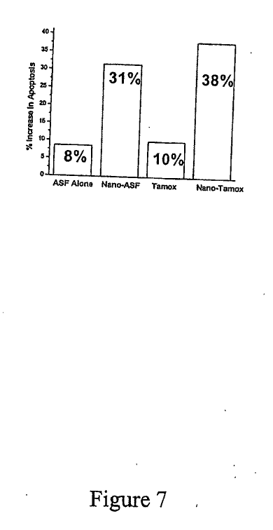

In a parallel experiment, an ASF microfluidized nanoemulsion was compared with a tamoxifen microfluidized nanoemulsion in their relative abilities to induce breast cancer cell apoptosis. Apoptosis was determined by two methods known in the art; the Vybrant Apoptosis Assay Kit® (Invitrogen, Carlsbad Calif.) and the DAP-1 Antibody Assay® (Novus, Littleton Colo.). Pooled data clearly show that ASF and tamoxifen were both up to four (4) times more efficacious in inducing apoptosis when delivered as a microfluidized nanoemulsion. Additionally, ASF was also observed a equally efficacious as tamoxifen in inducing cancer cell apoptosis over control levels. See FIG. 7.

### Example 8

### Improved Membrane Permeability Using Microfluidized Nanoemulsions

This example presents exemplary data showing that microfluidized nanoemulsions, as contemplated herein, substantially improves the membrane permeability of δ-tocopherol.

A microfluidized nanoemulsion containing 4.62 mg/ml δ-tocopherol was prepared according to Example 3. A non-microfluidized nanoemulsion containing 4.62 mg/ml δ-tocopherol was prepared as follows. To 240 mls (8 oz) of water heated to 60 degrees C. for 5 minutes was added 10 g canola oil, 1.2 g of δ-tocopherol and 10 grams Tween 80. The mixture was stirred for 20 minutes. Then 8 mls (8 g) of above mixture was mixed with 4 g of anhydrous Vanishing Creme/lotion (pharmacist grade).

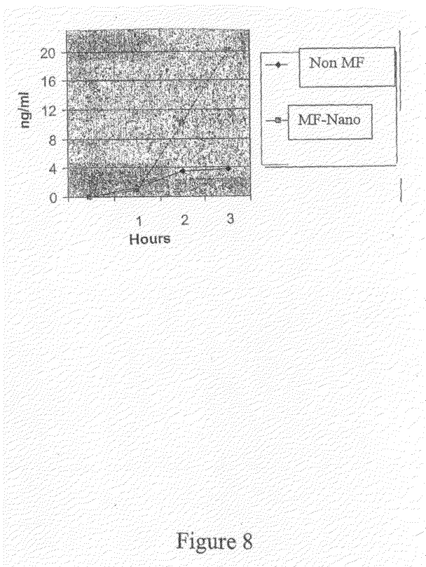

Membrane absorption was determined by applying each nanoemulsion preparation to a 1 in² shaven area on the back of a hamster (N=5). Systemic delivery of the absorbed 8-tocopherol was determined by HPCL using plasma sample extracts collected at zero time, 1 hour, 2 hours, and 3 hours post-application. These data show a progressive increase in the difference between non-microfluidized and microfluidized δ-tocopherol nanoemulsion (i.e., See Tables 8, 9 & 10, respectively and FIG. 8). The difference seen after three hours represents a 6-fold increase in membrane permeability of δ-tocopherol.

TABLE 8
δ-Tocopherol Plasma Levels After 1 Hour Of Membrane Absorption

| Treatment | Mean | Standard Error |
|---|---|---|
| Non-Microfluidized | 1.349 | 0.488 |
| Nanoemulsion | | |
| Microfluidized Nanoemulsion | 0.859 | 0.249 |
| Statistics | t = 0.918 df = 4 | p = 0.411 |

TABLE 9
δ-Tocopherol Plasma Levels After 2 Hours Of Membrane Absorption

| Treatment | Mean | Standard Error |
|---|---|---|
| Non-Microfluidized | 3.514 | 1.253 |
| Nanoemulsion | | |
| Microfluidized Nanoemulsion | 10.139 | 2.32 |
| Statistics | t = −3.260 df = 4 | p = 0.031 |

TABLE 10
δ-Tocopherol Plasma Levels After 3 Hours Of Membrane Absorption

| Treatment | Mean | Standard Error |
|---|---|---|
| Non-Microfluidized | 3.833 | 1.410 |
| Nanoemulsion | | |
| Microfluidized Nanoemulsion | 20.141 | 5.341 |
| Statistics | t = −3.793 df = 4 | p = 0.019 |

The data clearly demonstrate that microfluidized nanoemulsions, as contemplated herein, have a significantly improved membrane permeability when compared to traditional nanoemulsion preparations. Consequently, uniform microfluidized nanoemulsions have the capability of improved absorption and transfer into biological cells when compared to traditional nanoemulsion preparations.

1. A nanoemulsion comprising an anti-oxidant formulation, wherein said formulation comprises a cell-impermeant anti-oxidant, a cell permeant anti-oxidant, and a phospholipid.

2. The nanoemulsion of claim 1, wherein said nanoemulsion comprises a population of particles having diameters between approximately 10 and approximately 110 nanometers, wherein said nanoemulsion is not contaminated by particles having diameters larger than 110 nanometers.

3. The nanoemulsion of claim 2, wherein said particles encapsulate said formulation.

4. The nanoemulsion of claim 1, wherein said cell-impermeant anti-oxidant comprises tocopherol

5. The nanoemulsion of claim 1, wherein said cell-permeant anti-oxidant comprises sodium pyruvate.

6. The nanoemulsion of claim 1, wherein said phospholipid comprises phosphatidylcholine.

7. A nanoemulsion comprising tocopherol, wherein said nanoemulsion comprises a population of particles having diameters between approximately 10 and approximately 110 nanometers, wherein said nanoemulsion is not contaminated by particles having diameters larger than 110 nanometers.

8. A method, comprising;
a) providing;
i) a patient, wherein said patient exhibits at least one cancer symptom;
ii) a nanoemulsion comprising an anti-oxidant formulation, wherein said formulation comprises a cell-impermeant anti-oxidant, a cell permeant anti-oxidant, and a phospholipid;
b) delivering said nanoemulsion to said patients under conditions such that said at least one symptom is reduced.

9. The method of claim 8, wherein said nanoemulsion comprises a population of particles encapsulating said compound, wherein said particles having diameters between approximately 10 and approximately 110 nanometers, wherein said nanoemulsion is not contaminated by particles having diameters larger than 110 nanometers.

10. The method of claim 8, wherein said cancer symptom is caused by a neuroblastoma tumor.

11. The method of claim 8, wherein said cancer symptom is caused by a breast cancer tumor.

12. The method of claim 8, wherein said delivering comprises intra-tumoral.

13. The method of claim 8, wherein said delivering comprises a method selected from the group consisting of oral, transdermal, intravenous, intraperitoneal, intramuscular, and subcutaneous.

14. The method of claim 8, wherein said cell-impermeant anti-oxidant comprises a tocopherol.

15. The nanoemulsion of claim 8, wherein said cell-permeant anti-oxidant comprises sodium pyruvate.

16. The nanoemulsion of claim 8, wherein said phospholipid comprises phosphatidylcholine.

17. A method, comprising;
a) providing;
i) a patient, wherein said patient exhibits at least one cancer symptom;
ii) a uniform microfluidized nanoemulsion comprising tocopherol;
b) delivering said nanoemulsion to said patients under conditions such that said at least one symptom is reduced.

18. The method of claim 17, wherein said delivering comprises systemic.

19. The method of claim 17, wherein said delivering comprises intratumoral.

20. The method of claim 17, wherein said nanoemulsion further comprises a chemotherapeutic compound.
